(19) United States
(12) Patent Application Publication
(10) Pub. No.: US 2007/0079837 A1
(43) Pub. Date: Apr. 12, 2007

(54) Power nap

(76) Inventor: Jonathan Husni, Bentleyville, OH (US)

Correspondence Address: Jonathan Husni, 165 Gray Fox Run, Bentleyville, OH, 44022, US

(21) Appl. No.: 11/542,255

(22) Filed: Oct. 4, 2006

Related U.S. Application Data
(60) Provisional application No. 60/724,900, filed on Oct. 11, 2005.

(51) Int. Cl. A61B 19/00

(52) U.S. Cl. 128/898

(57) ABSTRACT

A method for inducing relaxation and sleep utilizing patterns and frequencies of audible tones to lead the brain into and out of natural cycles in a short period of time.

### RELATED APPLICATION

The present U.S. patent application is a continuation of Provisional Application Ser. No. 60/724,900 filed on Oct. 11, 2005.

### BACKGROUND OF THE INVENTION

It is well known that achieving a state of relaxation and sleep can be facilitated by listening to low frequency sounds within the frequency range of those brain waves which occur naturally when the brain is in a relaxed state, e.g. within a frequency range of about 5 to 12 hertz. Apparatus for generating such audible sounds and for presenting them in particular patterns are well known and obvious to those skilled in the electronic arts, and will therefore not be specifically described. Such patterns of tones can obviously be generated during use, e.g. by a micro-computer device with timing and tone generating circuitry, or can be pre-recorded and played back when desired.

### FIELD OF THE INVENTION

The present invention relates to inducing relaxation and sleep by means of audible tones within the range of relaxing brain wave frequencies and, more particularly, to inducing such relaxation and sleep by exposing a human subject to particular patterns of such tones to produce a short but highly relaxing nap, sometimes referred to as a “power nap.”

### SUMMARY OF THE INVENTION

The present invention is directed not simply to inducing relaxation and sleep by means of audible tones generally within the relaxed brain frequency range, but, rather to using particular types of patterns of such tones to induce power naps of generally short duration but of a highly relaxing nature. In general, this involves the discovery that such naps can be effectively induced by exposing a person to a series of such tones that gradually decrease in frequency from an initial tone near the top of the relaxed brain frequency range to an intermediate tone near the bottom of that range to induce restful sleep and then gradually increasing the frequency to a final tone with a frequency at least as high as that of the initial tone to bring the person back to a state of wakefulness. Sub-patterns of such total frequencies can also be beneficially included in the overall series of tones, for example by temporarily raising and lowering the frequency of individual tones.

### DETAILED DESCRIPTION OF AN ILLUSTRATIVE PREFERRED EMBODIMENT

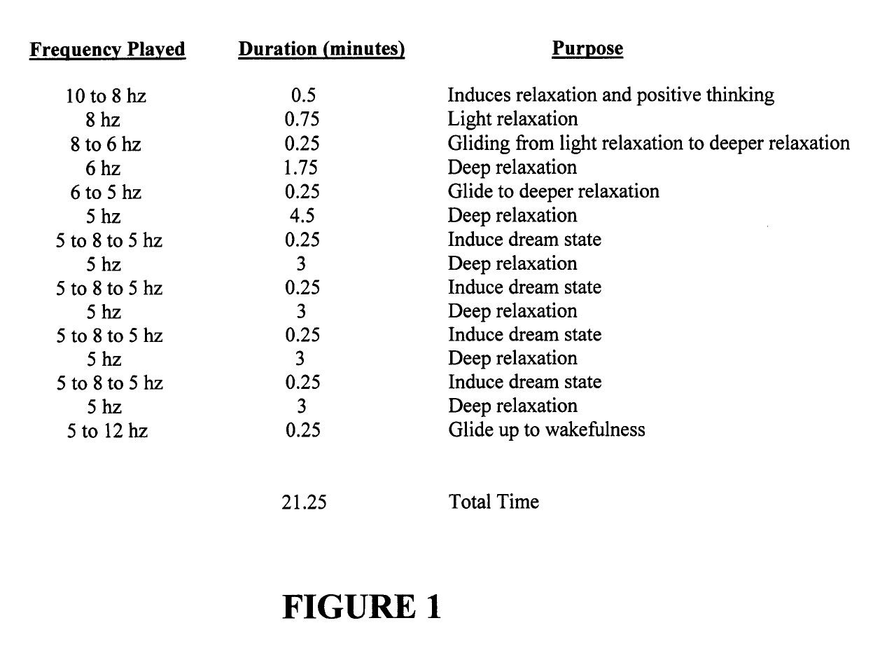

FIG. 1 illustrates an illustrative preferred embodiment of the invention in which a power nap slightly longer than 21 minutes is induced by having a person listen to a series of audible tones of the specific durations, starting at an initial frequency of about 10 hertz and declining in frequency to an intermediate frequency of about 5 hertz to final frequency of about 12 hertz to bring the person back into a state of wakefulness. Note also that temporary raising and lowering of the tone frequency, e.g. 5 to 8 to 5 hertz, occurs a number of times in the illustrative tonal series. The states of relaxation, sleep and wakefulness achieved at various stages are indicated to the right of the table, but it should be understood that such states are representative only and not specific correlations.

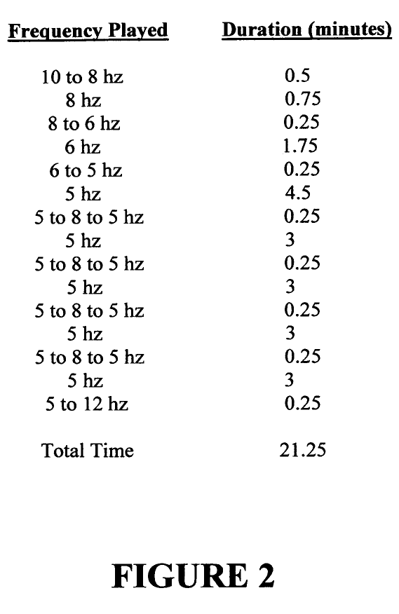

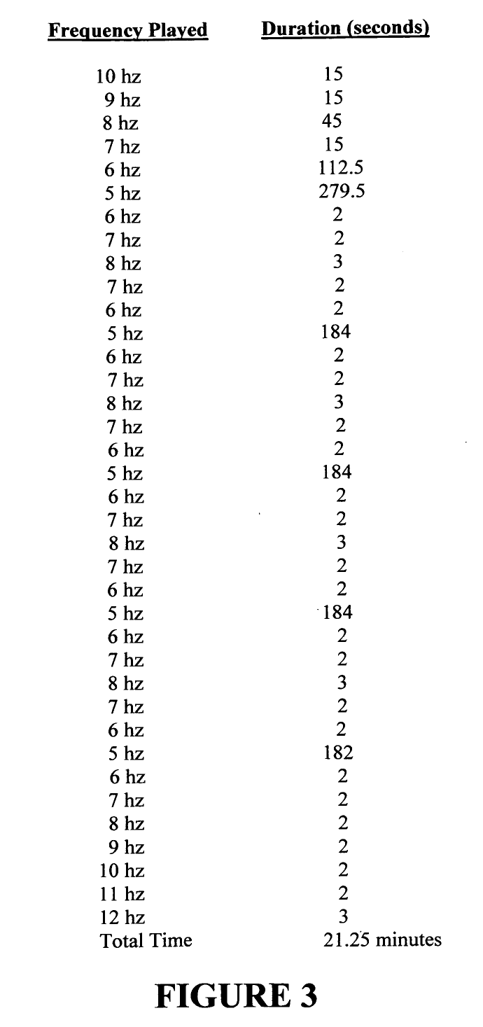

FIGS. 2 and 3 correspond generally to FIG. 1, but shows the various tonal frequencies and time durations of FIG. 1 in greater detail.

Although the invention has been described in conjunction with a preferred illustrative embodiment thereof, it should be understood that this description is intended to illustrate and not limit the scope of the invention, which is defined by the following claims.

What is claimed is:

1. The method of inducing a power nap in a human subject comprising exposing the subject to a series of audible tones of various predetermined durations starting with an initial frequency near the upper end of the relaxed brain wave frequencies, gradually reducing the frequencies of such tones to an intermediate frequency near the lower end of such relaxed brain frequencies to induce restful sleep, and then gradually increasing the frequencies of such tones to a final frequency at least as high as that of said initial frequency to restore the person to a state of wakefulness.

2. The method according to claim 1 in which said initial frequency is approximately 10 hertz, said intermediately frequency is approximately 5 hertz and said final frequency is approximately 12 hertz.

3. The method according to claim 1 in which at least one of said audible tones temporarily increases in frequency and then returns to its former frequency.

4. The method according to claim 1 in which said final frequency is higher than said initial initial frequency.
